(19) United States
(12) Patent Application Publication
(10) Pub. No.: US 2016/0361029 A1
(43) Pub. Date: Dec. 15, 2016

(54) BLOOD PRESSURE MEASURING APPARATUS BASED ON MULTIPROCESSING AND METHOD OF OPERATING THE SAME

(71) Applicant: SAMSUNG ELECTRONICS CO., LTD., Suwon-si (KR)

(72) Inventors: Jae Min KANG, Seoul (KR); Yong Joo KWON, Yongin-si (KR); Sun Kwon KIM, Suwon-si (KR)

(73) Assignee: SAMSUNG ELECTRONICS CO., LTD., Suwon-si (KR)

(21) Appl. No.: 15/158,030

(22) Filed: May 18, 2016

(30) Foreign Application Priority Data
Jun. 12, 2015 (KR) 10-2015-0083616

(51) Int. Cl. A61B 5/00; A61B 5/021

(52) U.S. Cl. CPC A61B 5/7282; A61B 5/02108; A61B 5/0004; A61B 5/742; A61B 5/7455; A61B 5/7405; A61B 5/7475; A61B 5/7203

(57) ABSTRACT

A blood pressure measuring apparatus based on multiprocessing and a method of operating the same are provided. The blood pressure measuring apparatus based on multiprocessing includes: at least two multichannel pulse wave measurers including a plurality of pulse wave measuring sensors configured to measure pulse waves; and at least two processors, each processor of the at least two processors being configured to receive corresponding pulse waves among the measured pulse waves from a corresponding multichannel pulse wave measurer of the at least two multichannel pulse wave measurers, and analyze the corresponding pulse waves and extract feature points from the analyzed pulse waves, wherein the at least two processors comprise a master processor configured to measure a blood pressure based on the feature points extracted by each of the at least two processors.

### CROSS-REFERENCE TO RELATED APPLICATION

This application claims priority from Korean Patent Application No. 10-2015-0083616, filed on Jun. 12, 2015 in the Korean Intellectual Property Office, the disclosure of which is incorporated herein by reference in its entirety.

### BACKGROUND

1. Field

Apparatuses and methods consistent with exemplary embodiments relate to a blood pressure measuring technology based on multiprocessing.

2. Description of the Related Art

With a growing interest in personal health, various types of biometric information detection devices are being developed, and devices specifically designed for healthcare are being developed with the widespread use of various wearable devices that may be directly worn by subjects.

A cuff-less blood pressure sensor is a blood pressure sensor of an indirect measurement method, in which blood pressure is measured by a Pulse Transit Time (PTT) method using an optical signal and an electrocardiogram (ECG) signal, or by a Pulse Wave Analysis (PWA) method that analyzes pulse waves based on an optical signal.

However, the PTT method is cumbersome in that touches of both hands are required, and an ECG signal is further needed in addition to a pulse wave signal. The PWA method analyzes only a waveform of pulse waves, and thus blood pressure may not be measured accurately by the method.

### SUMMARY

Exemplary embodiments address at least the above problems and/or disadvantages and other disadvantages not described above. Also, the exemplary embodiments are not required to overcome the disadvantages described above, and may not overcome any of the problems described above.

According to an aspect of an exemplary embodiment, there is provided a blood pressure measuring apparatus based on multiprocessing, the apparatus including: at least two multichannel pulse wave measurers including a plurality of pulse wave measuring sensors configured to measure pulse waves; and at least two processors, each processor of the at least two processors being configured to receive corresponding pulse waves among the measured pulse waves from a corresponding multichannel pulse wave measurer of the at least two multichannel pulse wave measurers, and analyze the corresponding pulse waves and extract feature points from the analyzed pulse waves, wherein the at least two processors comprise a master processor configured to measure a blood pressure based on the feature points extracted by each of the at least two processors.

The plurality of pulse wave measuring sensors may be arranged in a matrix or in a circle.

The plurality of pulse wave measuring sensors may measure the pulse waves by emitting light to a subject and by sensing light from the subject. The plurality of pulse wave measuring sensors may be further configured to emit light to a subject that reflects the light, and sense the light reflected from the subject and measure the pulse waves from the reflected light.

The master processor may determine a radial artery line, corresponding to a position of a radial artery of the subject, based on the pulse waves measured by the at least two multichannel pulse wave measurers, and measure the blood pressure based on a first feature point of a first pulse wave measured at a first point on the determined radial artery line and a second feature point of a second pulse wave measured a second point on the determined radial artery line.

The master processor may determine a pulse wave velocity between the first feature point and the second feature point, and measure the blood pressure based on the determined pulse wave velocity and a blood pressure estimation equation.

The master processor may determine a time difference between the first feature point and the second feature point, and determine the pulse wave velocity by dividing a distance between the first point and the second point by the determined time difference.

The at least two processors may further include a slave processor configured to transmit information on the corresponding pulse waves measured by the corresponding multichannel pulse wave measurer and information on the feature points extracted from the corresponding pulse waves to the master processor, and enter a sleep mode.

The master processor may generate a time synchronization signal to synchronize pulse wave measurement by each of the multichannel pulse wave measurers and pulse wave analysis by each of the processors, which are performed in parallel.

The blood pressure measuring apparatus based on multiprocessing may further include a user interface to output the measured blood pressure.

The blood pressure measuring apparatus based on multiprocessing may further include a communicator to transmit the measured blood pressure to an external device.

The blood pressure measuring apparatus based on multiprocessing may be configured to be wearable by the subject.

According to an aspect of another exemplary embodiment, there is provided a method of operating a multiprocessing-based blood pressure measuring apparatus including at least two multichannel pulse wave measurers that include a plurality of pulse wave measuring sensors, and at least two processors. The method includes: measuring first pulse waves by a first pulse wave measurer of the at least two multichannel pulse wave measurers; measuring second pulse waves by a second pulse wave measurer of the at least two multichannel pulse wave measures; analyzing the first pulse waves and extracting feature points from the first pulse waves by a first processor of the at least two processors; analyzing the second pulse waves and extracting feature points from the second pulse waves by a second processor of the at least two processors; and measuring, by the first processor, a blood pressure based on the feature points extracted by the first processor and the second processor, the first processor being operated as a master processor.

The plurality of pulse wave measuring sensors may be arranged in a matrix or in a circle.

The measuring the blood pressure may include: determining a radial artery line, corresponding to a position of a radial artery of a subject, based on the first pulse waves and the second pulse waves; and measuring the blood pressure based on a first feature point of pulse waves measured at a first point on the determined radial artery line and a second feature point of pulse waves measured a second point on the determined radial artery line.

The measuring the blood pressure may include: determining a pulse wave velocity between the first feature point and the second feature point; and measuring the blood pressure based on the determined pulse wave velocity and a blood pressure estimation equation.

The calculating the pulse wave velocity may include: determining a time difference between the first feature point and the second feature point; and determining the pulse wave velocity by dividing a distance between the first point and the second point by the determined time difference. The method may further include: transmitting information on the second pulse waves and information on the feature points extracted from the second pulse waves from the second processor to the first processor, the second processor being operated as a slave processor; and enabling the second processor to enter a sleep mode.

The method may further include: generating a time synchronization signal to synchronize pulse wave measurement by each of the multichannel pulse wave measurers and pulse wave analysis by each of the processors, which are performed in parallel.

### BRIEF DESCRIPTION OF THE DRAWINGS

The above and/or other aspects will be more apparent by describing certain exemplary embodiments, with reference to the accompanying drawings, in which:

FIG. 1 is a block diagram illustrating an example of a blood pressure measuring apparatus based on multiprocessing;

FIG. 2A is a diagram illustrating an example of an arrangement of pulse wave measuring sensors, and FIG. 2B is a diagram illustrating an example of pulse waves measured by using the pulse wave measuring sensors illustrated in FIG. 2A;

FIG. 3A is a diagram illustrating another example of an arrangement of pulse wave measuring sensors, and FIG. 3B is a diagram illustrating an example of pulse waves measured by using each of the pulse wave measuring sensors illustrated in FIG. 3A;

FIG. 4 is a diagram explaining a principle of calculating a pulse wave velocity by using feature points;

FIG. 5 is a block diagram illustrating another example of a blood pressure measuring apparatus based on multiprocessing; and

FIG. 6 is a flowchart illustrating a method of operating a blood pressure measuring apparatus 100 based on multiprocessing.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

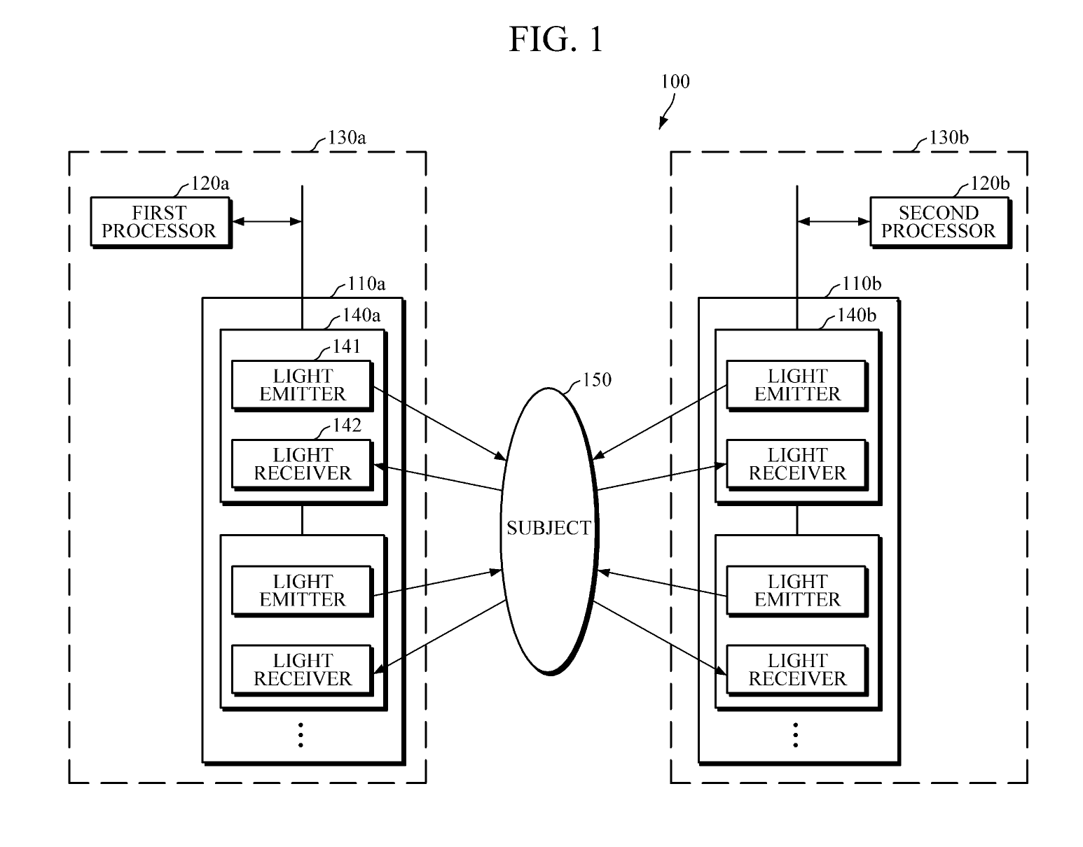

FIG. 1 is a block diagram illustrating an example of a blood pressure measuring apparatus based on multiprocessing.

The blood pressure measuring apparatus 100 may measure blood pressure of a subject in a non-invasive manner. For example, the blood pressure measuring apparatus 100 is a cuff-less type blood pressure measuring apparatus, which measures blood pressure by emitting light to a subject (e.g., a body part of a user wearing the blood pressure measuring apparatus 100), measuring pulse waves by sensing reflected or scattered light, and analyzing the measured pulse waves.

The blood pressure measuring apparatus 100 may be a wearable device to be worn on a subject. For example, the blood pressure measuring apparatus 100 may be a wristwatch type, a bracelet type, or a wristband type. However, the blood pressure measuring apparatus 100 is not limited thereto, and may be a ring type, a glasses type, a hairband type, and the like.

Referring to FIG. 1, the blood pressure measuring apparatus 100 includes a first multichannel pulse wave measurer 110a, a second multichannel pulse wave measurer 110b, a first processor 120a, and a second processor 120b.

The first processor 120a may be configured as a master processor. Further, the first multichannel pulse wave measurer 110a and the first processor 120a may be interconnected with each other to form a first processing group 130a, and the second multichannel pulse wave measurer 110b and the second processor 120b may be interconnected with each other to form a second processing group 130b. When the first processor 120a operates as a master processor, the second processor 120b may operate as a slave processor.

Although the blood pressure measuring apparatus 100 is illustrated as including the two processing groups 130a and 130b in FIG. 1, the blood pressure measuring apparatus 100 may include three or more processing groups according to use and performance of a system. That is, each of the multichannel pulse wave measurer 110 and the processor 120 may be increased to three or more in number.

The multichannel pulse wave measurers 110a and 110b may measure pulse waves at various points. To this end, the first multichannel pulse wave measurer 110a may include a plurality of pulse wave measuring sensors 140a, and the second multichannel pulse wave measurer 110b may include a plurality of pulse wave measuring sensors 140b.

Each of the pulse wave measuring sensors 140a and 140b may include a light emitter 141 and a light receiver 142. The light emitter 141 may emit light on the subject 150, and the light receiver 142 may detect light scattered or reflected from the subject 150. The pulse wave measuring sensors 140a and 140b may acquire pulse waves from a detected optical signal.

That is, the first multichannel pulse wave measurer 110a may measure pulse waves at various points by using the plurality of pulse wave measuring sensors 140a, and the second multichannel pulse wave measurer 110b may measure pulse waves at various points by using the plurality of pulse wave measuring sensors 140b.

In one exemplary embodiment, a light emitting diode (LED) or a laser diode may be used as the light emitter 141. A photo diode, a photo transistor (PTr), or a charge-couple device (CCD) may be used as the light receiver 142.

The processors 120a and 120b may generate a driving signal to measure pulse waves by using the respective connected multichannel pulse wave measurers 110a and 110b. That is, the first processor 120a generates a driving signal to drive the first multichannel pulse wave measurer 110a, and the second processor 120b generates a driving signal to drive the second multichannel pulse wave measurer 110b.

However, the processors 120a and 120b are not limited thereto, and the first processor 120a, which is configured as a master processor, may generate both the driving signal to drive the first multichannel pulse wave measurer 110a and the driving signal to drive the second multichannel pulse wave measurer 110b.

The first multichannel pulse wave measurer 110a may measure pulse waves at various points by sequentially driving the pulse wave measuring sensors 140a according to the driving signal generated by the first processor 120a, and the second multichannel pulse wave measurer 110b may measure pulse waves by sequentially driving the pulse wave measuring sensors 140b according to the driving signal generated by the second processor 120b.

The processors 120a and 120b may analyze pulse waves measured by the respective connected multichannel pulse wave measurers 110a and 110b. That is, the first processor 120a analyzes pulse waves measured by the first multichannel pulse wave measurer 110a, and the second processor 120b analyzes pulse waves measured by the second multichannel pulse wave measurer 110b.

In one exemplary embodiment, the first processor 120a may extract feature points from each pulse wave measured by the pulse wave measuring sensors 140a of the first multichannel pulse wave measurer 110a. The second processor 120b may extract feature points from each pulse wave measured by the pulse wave measuring sensors 140b of the second multichannel pulse wave measurer 110b. The feature points may include a start point, a maximum point, a minimum point, and the like.

The first processor 120a, which is configured as a master processor, may receive pulse wave information and feature point information from the second processor 120b, and may measure blood pressure based on pulse waves measured by the first multichannel pulse wave measurer 110a, feature points extracted from the pulse waves, and pulse wave information and feature point information received from the second processor 120b.

The first processor 120a may determine a radial artery line, which corresponds to the position of a radial artery of the subject 150, based on pulse waves measured by the first multichannel pulse wave measurer 110a and pulse wave information received from the second processor 120b. More specifically, when the multichannel pulse wave measurers 110a and 110b measure pulse waves, a signal-to-noise ratio of pulse waves measured by the pulse wave measuring sensors 140a and 140b may vary depending on a relative distance between locations of the pulse wave measuring sensors 140a and 140b and the radial artery of the subject 150. For example, pulse waves measured by one pulse wave measuring sensor, which is located relatively closer to the radial artery among the plurality of pulse wave measuring sensors 140a and 140b, have a higher signal-to-noise ratio, and pulse waves measured by one pulse wave measuring sensor, which is located relatively farther from the radial artery among the plurality of pulse wave measuring sensors 140a and 140b, have many noise components, and thus have a lower signal-to-noise ratio. As described above, by analyzing a signal-to-noise ratio of measured pulse waves, at least two pulse wave measuring sensors which have a relatively higher signal-to-noise ratio may be selected from among the plurality of pulse wave measuring sensors 140a and 140b, so that a radial artery line may be determined by connecting location points of the two pulse wave measuring sensors.

The first processor 120a, configured as a master processor, may measure blood pressure by using feature points of pulse waves measured at two points on the determined radial artery line. For example, assuming that the first pulse wave is measured at a first point on the radial artery line, and the second pulse wave is measured at a second point on the radial artery line, the first processor 120a may calculate a pulse wave velocity by calculating a time difference Δt between a feature point of the first pulse wave and a feature point of the second pulse wave, and by dividing a distance between the first point and the second point, i.e., a distance between a pulse wave measuring sensor measuring the first pulse wave and a pulse wave measuring sensor measuring the second pulse wave, by the calculated time difference Δt.

The first processor 120a may calculate blood pressure by using the pulse wave velocity and a blood pressure estimation equation that defines a relationship between blood pressure and the pulse wave velocity, and the blood pressure estimation equation may be stored in a database or in an external memory.

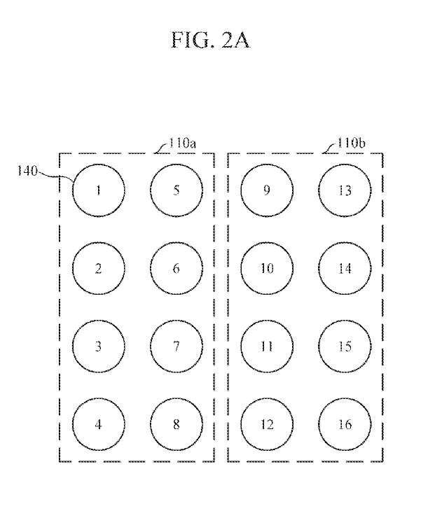

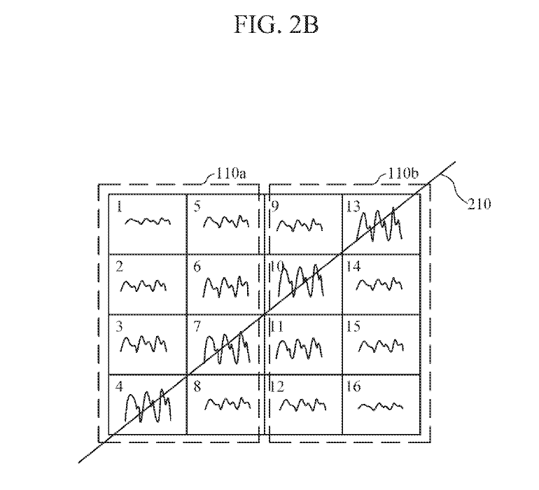

The first processor 120a, configured as a master processor, may generate a time synchronization signal for time synchronization of pulse wave measurement and/or pulse wave analysis with the second processor 120b, and may transmit the generated time synchronization signal to the second processor 120b. Upon receiving the time synchronization signal, the second processor 120b is time-synchronized with the first processor 120a, so that the second processor 120b may measure and/or analyze pulse waves in parallel with the first processor 120a. Unlike the example illustrated in FIG. 2, in the case where the blood pressure measuring apparatus 100 includes three processing groups 130, i.e., in the case where each of the multichannel pulse wave measurer 110 and the processor 120 is increased to three or more in number, the master processor may transmit the time synchronization signal to processors other than the master processor.

After finishing measurement of pulse waves and extraction of feature points, the second processor 120b, which is not a master processor, may transmit information on the measured pulse waves and extracted feature points to the first processor 120a which is a master processor. Further, upon completing transmission of information to the first processor 120a, the second processor 120b may enter a sleep mode. The sleep mode is a power saving mode, in which a minimum power is supplied or the power supply is blocked such that processors stop operating. Unlike the example illustrated in FIG. 2, in the case where the blood pressure measuring apparatus 100 includes three processing groups 130, i.e., each of the multichannel pulse wave measurer 110 and the processor 120 is increased to three or more in number, processors other than the master processor may enter a sleep mode after completing transmission of information to the master processor.

The subject 150 is a subject of which blood pressure is to be measured, and may be a body part that may contact or may be adjacent to the pulse wave measuring sensors 140a and 140b of the blood pressure measuring apparatus 100, or a body part of which pulse waves may be easily measured by using photoplethysmography (PPG). For example, the subject 150 may be an area on a wrist that is adjacent to the radial artery. In the case of measuring pulse waves on a position of the wrist over the radial artery, there may be relatively less external factors, such as the thickness of the skin tissue of the wrist, which may cause measurement errors. The radial artery is known to be a position where blood pressure may be measured more accurately than other arteries. However, the subject 150 is not limited thereto, and may be distal body portions, such as fingers and toes, which have a high density of blood vessels.

FIG. 2A is a diagram illustrating an example of an arrangement of pulse wave measuring sensors, and FIG. 2B is a diagram illustrating an example of pulse waves measured by using the pulse wave measuring sensors illustrated in FIG. 2A.

Referring to FIGS. 1 and 2A, 16 pulse wave measuring sensors 140 may be arranged in a 4×4 matrix, in which first to eighth pulse wave measuring sensors may be included in the first multichannel pulse wave measurer 110a, and a ninth to sixteenth pulse wave measuring sensors may be included in the second multichannel pulse wave measurer 110b. As described above, each of the pulse wave measuring sensors 140 includes a light emitter and a light receiver.

Each of the pulse wave measuring sensors 140 measures pulse waves by using its own light emitter and a light receiver. FIG. 2B illustrates an exemplary embodiment of pulse waves measured by using each of the pulse wave measuring sensors illustrated in FIG. 2A.

The first processor 120a analyzes first to eighth pulse waves measured by the first to eighth pulse wave measuring sensors, and extracts feature points thereof. The second processor 120b analyzes ninth to sixteenth pulse waves measured by the ninth to sixteenth pulse wave measuring sensors, and extracts feature points thereof.

The second processor 120b transmits information on the ninth to sixteenth pulse waves, and information on feature points extracted from the ninth to sixteenth pulse waves to the first processor 120a which is a master processor.

The first processor 120a may determine a radial artery line by analyzing a signal-to-noise ratio of each pulse wave based on the information on the first to sixteenth pulse waves. In the exemplary embodiment, a radial artery line is set as a line 210 determined by connecting location points of the fourth, seventh, tenth, and thirteenth pulse wave measuring sensors that measure pulse waves (fourth, seventh, tenth, and thirteenth pulse waves), which have a relatively higher signal-to-noise ratio.

The first processor 120a calculates blood pressure by using feature points of any two pulse waves (e.g., the fourth and thirteenth pulse waves) among the fourth, seventh, and thirteenth pulse waves.

In the case where the blood pressure measuring apparatus 100 includes four processing groups 130, i.e., in the case where each of the multichannel pulse wave measurer 110 and the processor 120 is four in number, the first, second, fifth, and sixth pulse wave measuring sensors may be included in the first multichannel pulse wave measurer; the third, fourth, seventh, and eighth pulse wave measuring sensors may be included in the second multichannel pulse wave measurer; the ninth, tenth, thirteenth, and fourteenth pulse wave measuring sensors may be included in the third multichannel pulse wave measurers; and the eleventh, twelfth, fifteenth, and sixteenth pulse wave measuring sensors may be included in the fourth multichannel pulse wave measurer. In this case, pulse waves measured by the first multichannel pulse wave measurer may be analyzed by the first processor; pulse waves measured by the second multichannel pulse wave measurer may be analyzed by the second processor; pulse waves measured by the third multichannel pulse wave measurer may be analyzed by the third processor; and pulse waves measured by the fourth multichannel pulse wave measurer may be analyzed by the fourth processor.

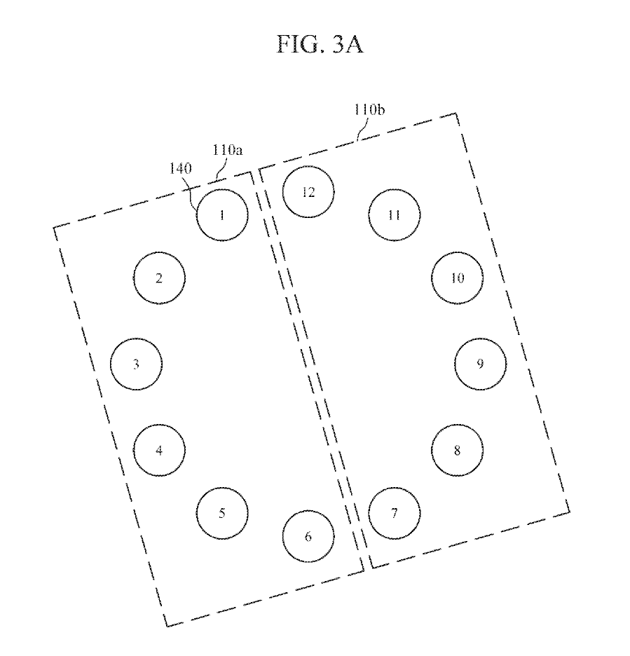

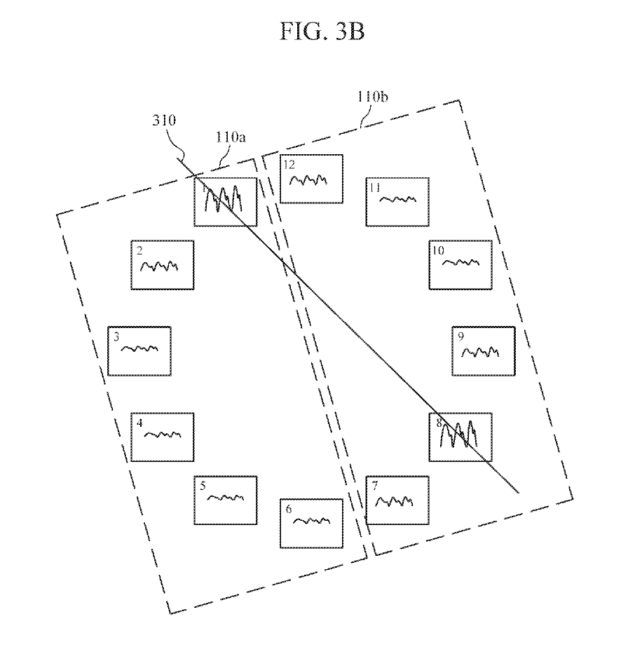

FIG. 3A is a diagram illustrating another example of an arrangement of pulse wave measuring sensors, and FIG. 3B is a diagram illustrating an example of pulse waves measured by using each of the pulse wave measuring sensors illustrated in FIG. 3A.

Referring to FIGS. 1 and 3A, 12 pulse wave measuring sensors 140 may be arranged in a circle, in which first to sixth pulse wave measuring sensors may be included in the first multichannel pulse wave measurer 110a, and seventh to twelfth pulse wave measuring sensors may be included in the second multichannel pulse wave measurer 110b. As described above, each of the pulse wave measuring sensors 140 includes a light emitter and a light receiver.

Each of the pulse wave measuring sensors 140 measures pulse waves by using its own light emitter and light receiver. FIG. 3B illustrates an exemplary embodiment of pulse waves measured by using each of the pulse wave measuring sensors illustrated in FIG. 3A.

The first processor 120a analyzes first to sixth pulse waves measuring by the first to sixth pulse wave measuring sensors, and extracts feature points thereof. The second processor 120b analyzes seventh to twelfth pulse waves measured by the seventh to twelfth pulse wave measuring sensors, and extracts feature points thereof.

The second processor 120b transmits information on the seventh to twelfth pulse waves, and information on feature points extracted from the seventh to twelfth pulse waves to the first processor 120a which is a master processor.

The first processor 120a may form a radial artery line by analyzing a signal-to-noise ratio of each pulse wave based on the information on the first to twelfth pulse waves. In the exemplary embodiment, a radial artery line is set as a line 310 determined by connecting location points of the first and eighth pulse wave measuring sensors that measure pulse waves (first and eighth pulse waves), which have a relatively higher signal-to-noise ratio.

The first processor 120a calculates blood pressure by using feature points of the first and eighth pulse waves.

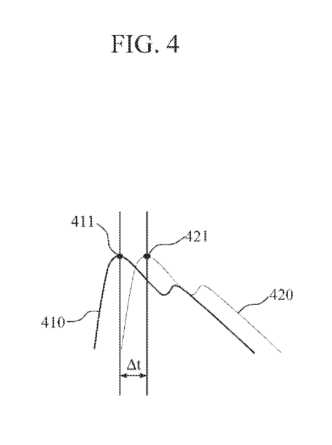

FIG. 4 is a diagram explaining a principle of calculating a pulse wave velocity by using feature points.

Pulse wave velocity refers to the velocity of pulse waves flowing through blood vessels, and may be calculated by extracting feature points 411 and 421 from pulse waves 410 and 420 measured at two points, by calculating a time difference Δt between the extracted feature points 411 and 421, and then by dividing a distance between measured points of the pulse waves 410 and 420, i.e., a distance between pulse wave measuring sensors that measure the pulse waves 410 and 420, by the calculated time difference Δt.

Since pulse wave velocity is increased when blood vessel elasticity is reduced, the pulse wave velocity may be a measure of change in blood vessel elasticity and blood pressure, and may be used to find a correlation between the pulse wave velocity and blood pressure values.

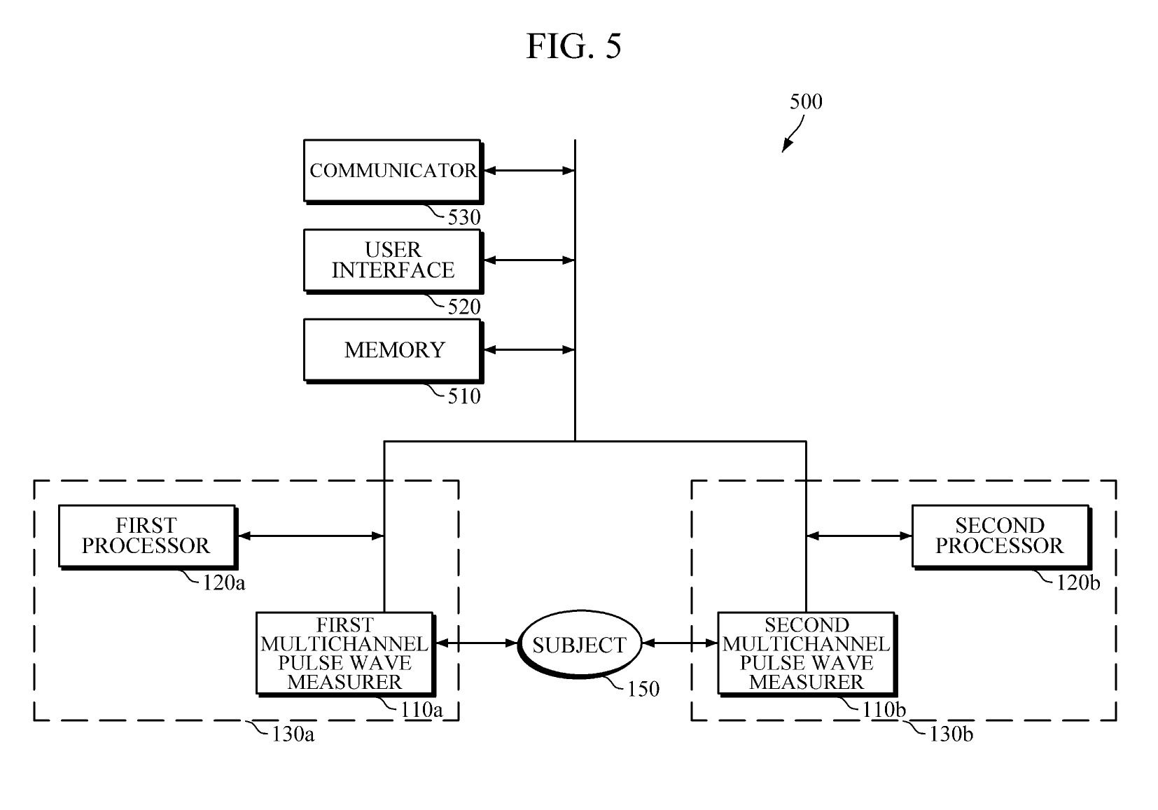

FIG. 5 is a block diagram illustrating another example of a blood pressure measuring apparatus based on multiprocessing.

Referring to FIG. 5, when compared to the blood pressure measuring apparatus 100, the blood pressure measuring apparatus 500 may further include a memory 510, a user interface 520, and a communicator 530 selectively.

The memory 510 may store programs to process and control the processors 120a and 120b, and may store input/output data. For example, the memory 510 may store programs for pulse wave analysis and blood pressure calculation performed by the processors 120a and 120b, and/or information on a blood pressure estimation equation. Further, the memory 510 may store pulse wave measurement results of the multichannel pulse wave measurer 110a and 110b. The processors 120a and 120b may read the pulse wave measurement results from the memory 510 and process the results.

The memory 510 may include at least one storage medium among flash memory type, hard disk type, multi-media card micro type, card type memory (e.g., SD or XD memory, etc.), random access memory (RAM), static random access memory (SRAM), read-only memory (ROM), electrically erasable programmable read only memory (EEPROM), programmable read only memory (PROM), magnetic memory, magnetic disks, optical discs, and the like.

The user interface 520 is an interface between the blood pressure measuring apparatus 500 and a user, and/or an interface between the blood pressure measuring apparatus 500 and other external device, and may include an input and an output. The user may be a subject of which blood pressure is to be measured, i.e., the subject 150, but may be a concept wider than the subject 150.

Information for operating the blood pressure measuring apparatus 500 is input through the user interface 520, and measurement results of blood pressure may be output through the user interface 520. The user interface 520 may include, for example, a button, a connector, a keypad, a display, and the like, and may further include a sound output component or a vibration motor.

The communicator 530 may communicate with external devices. For example, the communicator 530 may transmit measurement results of blood pressure to an external device, or may receive various types of information useful for measuring blood pressure from an external device.

The external device may be medical equipment using information on the measured blood pressure, a printer to print out results, or a display to display information on the measured blood pressure. In addition, the external device may be a smartphone, a mobile phone, a personal digital assistant (PDA) device, a laptop computer, a personal computer (PC), and other mobile or non-mobile computing devices.

The communicator 530 may communicate with external devices by using Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra Wideband (UWB) communication, Ant+communication, Wi-Fi communication, Radio Frequency Identification (RFID) communication, and the like. However, the communicator 530 is merely illustrative, and is not limited thereto.

Further, depending on the use and performance of a system, each of the processing groups 130a and 130b may include a local memory and a local communicator.

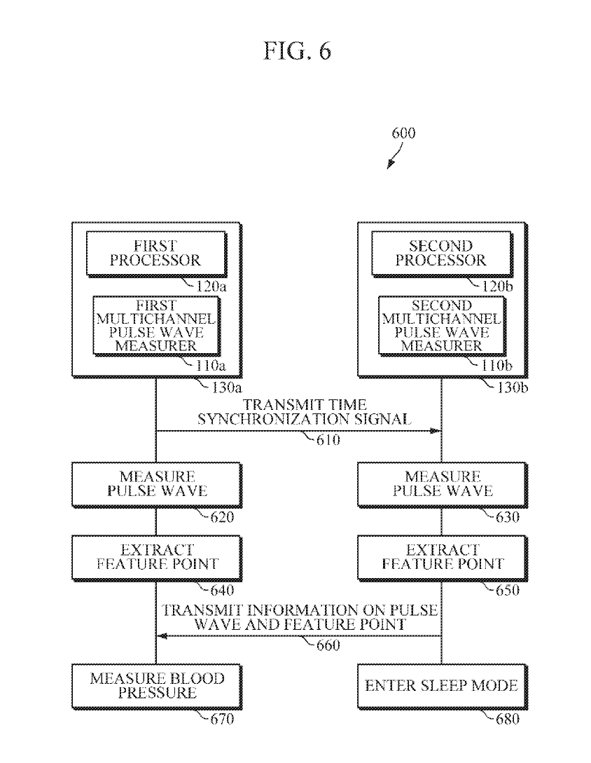

FIG. 6 is a flowchart illustrating a method of operating a blood pressure measuring apparatus 100 based on multiprocessing. The first processing group 130a includes the first multichannel pulse wave measurer 110a and the first processor 120a, and the second processing group 130a includes the second processor 120b. Further, it is assumed that the first processor 120a is configured as a master processor.

Referring to FIGS. 1 and 6, the first processor 120a generates a time synchronization signal for time synchronization of pulse wave measurement and/or pulse wave analysis with the second processor 120b, and transmits the generated time synchronization signal to the second processor 120b in operation 610.

According to a driving signal from the first processor 120a, the first multichannel pulse wave measurer 110a measures pulse waves at various points by sequentially driving a plurality of pulse wave measuring sensors 140a in operation 620. Further, according to a driving signal from the second processor 120b, the second multichannel pulse wave measurer 110b measures pulse waves at various points by sequentially driving a plurality of pulse wave measuring sensors 140b in operation 630. In this case, the pulse waves measured by the first multichannel pulse wave measurer 110a in operation 620 and the pulse waves measured by the second multichannel pulse wave measurer 110b in operation 630 may be time-synchronized according to the time synchronization signal, and thus may be performed in parallel.

The pulse wave measuring sensors included in the first multichannel pulse wave measurer 110a and the second multichannel pulse wave measurer 120a may be arranged in a matrix or in a circle.

The first processor 120a analyzes the pulse waves measured by the first multichannel pulse wave measurer 110a, which is connected with the first processor 120a, and extracts feature points from each of the pulse waves in operation 640. Further, the second processor 120b analyzes the pulse waves measured by the second multichannel pulse wave measurer 110b, which is connected with the second processor 120b, and extracts feature points from each of the pulse waves in operation 650. In this case, the pulse waves analyzed by the first processor 120a in operation 640 and the pulse waves analyzed by the second processor 120b in operation 650 may be time-synchronized according to the time synchronization signal, and thus may be performed in parallel.

The second processor 120b transmits information on pulse waves measured by the second multichannel pulse wave measurer 110b and information on feature points extracted from each pulse wave to the first processor 120a in operation 660.

The first processor 120a calculates blood pressure based on pulse waves measured by the first multichannel pulse wave measurer 110a, feature points extracted from the pulse waves, and pulse wave information and feature point information received from the second processor 120b in operation 670.

In one exemplary embodiment, the first processor 120a determines a radial artery line, corresponding to the location of the radial artery of the subject 150, based on pulse waves measured by the first multichannel pulse wave measurer 110a and pulse wave information received from the second processor 120b, and may calculate blood pressure by using feature points of pulse waves measured at two points on the determined radial artery line. For example, the first processor 120a may calculate a pulse wave velocity based on feature points of pulse waves measured at two points on the radial artery line, and may calculate blood pressure by using the calculated pulse wave velocity and a blood pressure estimation equation. The blood pressure estimation equation may be stored in a database or in an external memory.

The first processor 120a may calculate a pulse wave velocity by calculating a time difference Δt between feature points of pulse waves measured at two points, and by dividing a distance between the two points by the calculated time difference Δt.

Upon completing transmission of pulse wave information and feature point information in operation 660, the second processor 120b, which is not a master processor, enters a sleep mode in operation 680.

While not restricted thereto, an exemplary embodiment can be realized as a computer-readable code that is stored on a computer-readable recording medium and executed by a computer, a processor, integrated circuit, etc. Codes and code segments needed for realizing the present disclosure can be easily deduced by computer programmers of ordinary skill in the art. The computer-readable recording medium may be any type of recording device in which data is stored in a computer-readable manner. Examples of the computer-readable recording medium include a read-only memory (ROM), a random-access memory (RAM), a CD-ROM, a magnetic tape, a floppy disc, an optical disk, and the like. Further, the computer-readable recording medium can be distributed over a plurality of computer systems connected to a network so that a computer-readable recording medium is written thereto and executed therefrom in a decentralized manner. Also, an exemplary embodiment may be written as a computer program transmitted over a computer-readable transmission medium, such as a carrier wave, and received and implemented in general-use or special-purpose digital computers that execute the programs. Moreover, it is understood that in exemplary embodiments, one or more units of the above-described apparatuses and devices can include circuitry, a processor, a microprocessor, etc., and may execute a computer program stored in a computer-readable medium.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A blood pressure measuring apparatus based on multiprocessing, the apparatus comprising:
at least two multichannel pulse wave measurers including a plurality of pulse wave measuring sensors configured to measure pulse waves; and
at least two processors, each processor of the at least two processors being configured to receive corresponding pulse waves among the measured pulse waves from a corresponding multichannel pulse wave measurer of the at least two multichannel pulse wave measurers, and analyze the corresponding pulse waves and extract feature points from the analyzed pulse waves,
wherein the at least two processors comprise a master processor configured to measure a blood pressure based on the feature points extracted by each of the at least two processors.

2. The apparatus of claim 1, wherein the plurality of pulse wave measuring sensors are arranged in a matrix or in a circle.

3. The apparatus of claim 1, wherein the plurality of pulse wave measuring sensors are further configured to emit light to a subject that reflects the light, sense the light reflected from the subject and measure the pulse waves from the reflected light.

4. The apparatus of claim 1, wherein the master processor is further configured to determine a radial artery line, corresponding to a position of a radial artery of the subject, based on the pulse waves measured by the at least two multichannel pulse wave measurers, and measure the blood pressure based on a first feature point of a first pulse wave measured at a first point on the determined radial artery line and a second feature point of a second pulse wave measured a second point on the determined radial artery line.

5. The apparatus of claim 4, wherein the master processor is further configured to determine a pulse wave velocity between the first feature point and the second feature point, and measure the blood pressure based on the determined pulse wave velocity and a blood pressure estimation equation.

6. The apparatus of claim 5, wherein the master processor is further configured to determine a time difference between the first feature point and the second feature point, and determine the pulse wave velocity by dividing a distance between the first point and the second point by the determined time difference.

7. The apparatus of claim 1, wherein the at least two processors further comprise a slave processor configured to transmit information on the corresponding pulse waves measured by the corresponding multichannel pulse wave measurer and information on the feature points extracted from the corresponding pulse waves to the master processor, and enter a sleep mode.

8. The apparatus of claim 1, wherein the master processor is further configured to generate a time synchronization signal to synchronize pulse wave measurement by each of the multichannel pulse wave measurers and pulse wave analysis by each of the processors, which are performed in parallel.

9. The apparatus of claim 1, further comprising a user interface to output the measured blood pressure.

10. The apparatus of claim 1, further comprising a communicator to transmit the measured blood pressure to an external device.

11. The apparatus of claim 1, wherein the blood pressure measuring apparatus based on multiprocessing is configured to be wearable by the subject.

12. A method of operating a multiprocessing-based blood pressure measuring apparatus comprising at least two multichannel pulse wave measurers that include a plurality of pulse wave measuring sensors, and at least two processors, the method comprising:
measuring first pulse waves by a first pulse wave measurer of the at least two multichannel pulse wave measurers;
measuring second pulse waves by a second pulse wave measurer of the at least two multichannel pulse wave measures;
analyzing the first pulse waves and extracting feature points from the first pulse waves by a first processor of the at least two processors;
analyzing the second pulse waves and extracting feature points from the second pulse waves by a second processor of the at least two processors; and
measuring, by the first processor, a blood pressure based on the feature points extracted by the first processor and the second processor, the first processor being operated as a master processor.

13. The method of claim 12, wherein the plurality of pulse wave measuring sensors are arranged in a matrix or in a circle.

14. The method of claim 12, wherein the measuring the blood pressure comprises:
determining a radial artery line, corresponding to a position of a radial artery of a subject, based on the first pulse waves and the second pulse waves; and
measuring the blood pressure based on a first feature point of pulse waves measured at a first point on the determined radial artery line and a second feature point of pulse waves measured a second point on the determined radial artery line.

15. The method of claim 14, wherein the measuring the blood pressure comprises:
determining a pulse wave velocity between the first feature point and the second feature point; and
measuring the blood pressure based on the determined pulse wave velocity and a blood pressure estimation equation.

16. The method of claim 15, wherein the determining the pulse wave velocity comprises:
determining a time difference between the first feature point and the second feature point; and
determining the pulse wave velocity by dividing a distance between the first point and the second point by the determined time difference.

17. The method of claim 12, further comprising:
transmitting information on the second pulse waves and information on the feature points extracted from the second pulse waves from the second processor to the first processor, the second processor being operated as a slave processor; and
enabling the second processor to enter a sleep mode.

18. The method of claim 12, wherein further comprising generating a time synchronization signal to synchronize pulse wave measurement by each of the multichannel pulse wave measurers and pulse wave analysis by each of the processors, which are performed in parallel.
